(19) United States
(12) Patent Application Publication
(10) Pub. No.: US 2025/0035328 A1
(43) Pub. Date: Jan. 30, 2025

(54) BIOACTIVE COATING FOR INDOOR AIR QUALLITY IMPROVEMENT

(71) Applicants: UNIVERSITÉ DE MONS, Mons (BE); MATERIA NOVA, Mons (BE)

(72) Inventors: Anne-Lise HANTSON, Mont-Saint Guibert (BE); Cristiana CORDIERO DE CASTRO, Mont-Saint Guibert (BE); Tangi SENECHAL, Mont-Saint Guibert (BE); Driss LAHEM, Mont-Saint Guibert (BE); Mireille POELMAN, Mont-Saint Guibert (BE)

(73) Assignees: UNIVERSITÉ DE MONS, Mons (BE); MATERIA NOVA, Mons (BE)

(21) Appl. No.: 18/717,072

(22) PCT Filed: Dec. 16, 2022
(86) PCT No.: PCT/EP2022/086446

§ 371 Date: Jun. 6, 2024

(30) Foreign Application Priority Data
Dec. 17, 2021 (GB) 2118389.2

(51) Int. Cl. F24F 8/175; C12N 1/20; C12N 11/04; C12R 1/40

(52) U.S. Cl. CPC F24F 8/175; C12N 1/205; C12N 11/04; C12R 2001/40

(57) ABSTRACT

A method to reduce concentration of a VOC (volatile organic compound) in air in contact with a coated surface comprising: —providing a surface—encapsulation of whole microorganism of Pseudomonas putida in a sol-gel matrix—application of the obtained sol-gel matrix to the surface to form the coated surface—exposing the air to the coated surface.

The present invention relates to a method to reduce VOC (volatile organic compound) concentration in air in contact with a surface coated with a sol gel matrix comprising encapsulated microorganism of Pseudomonas putida and to the use of encapsulated microorganism of Pseudomonas putida in a sol-gel matrix to reduce the concentration of a VOC in air.

Indoor air pollution of residential units and workplaces is a major concern nowadays. Toxic pollutants such as formaldehyde, which may have carcinogenic effects in health, are constantly released from distinct construction and decoration materials and/or household products. The development of bioactive coatings incorporating biomolecules able to capture and degrade these toxic compounds is of major interest. However, the conservation of their bioactivity is crucial throughout time. The incorporation of whole cells of Pseudomonas putida in the sol-gel matrix, compared to extracted and purified enzymes, can provide an optimized environment allowing the conservation of enzyme stability and co-factors regeneration, needed for the enzymatic conversion, besides eliminating extraction/purification costs.

In accordance with one aspect as defined in claim 1, the present invention provides a method to reduce the concentration of a VOC (volatile organic compound) in air in contact with a coated surface comprising:
providing a surface
encapsulation of whole microorganisms of Pseudomonas putida in a sol-gel matrix
application of the obtained sol-gel matrix to the surface to form the coated surface and
exposing the air to the coated surface.

The dependent claims define preferred or alternative embodiments.

In accordance with another aspect as defined in claim 15, the present invention provides use of encapsulated whole microorganism of Pseudomonas putida in a sol-gel matrix to reduce the concentration of a VOC in air.

Any feature described herein in relation to a particular aspect of the invention may be used in relation to any other aspect of the invention. Notably, any aspect described in relation to the method to reduce the concentration of a VOC (volatile organic compound) in air equally applies to the use of encapsulated whole microorganism of Pseudomonas putida in a sol-gel matrix to reduce the concentration of a VOC in air.

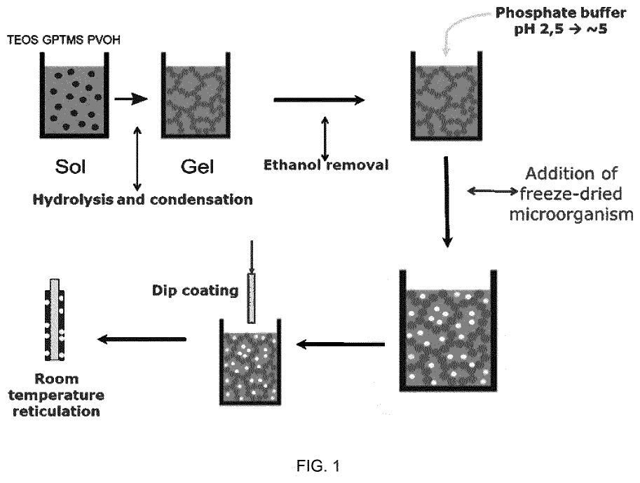

FIG. 1 shows a scheme of a preferred embodiment in accordance with the present invention.

Volatile organic compounds (VOCs) are any organic compound emanating from human activities, other than methane, which are capable of producing photochemical oxidants by reacting with nitrogen oxide in the presence of sunlight and having at 293, 15 K a vapour (European Union in directives 1999/13/CE and 2008/50/CE and Code of Federal Regulations, 40: Chapter 1, Subchapter C, Part 51, Subpart F, 51100) or VOCs are organic chemicals that have a high vapour pressure at room temperature. VOCs as commonly defined are any organic compound having an initial boiling point less than or equal to 250° C. (482° F.) measured at a standard atmospheric pressure of 101.3 kPa. VOCs comprise an important group of chemicals that evaporate easily at room temperature and are commonly present in indoor air. Some of them may cause short- and/or long-term adverse health effects. The VOCs of interest in the present invention are the polluting compounds present in the indoor air of buildings and the aim of the invention is to degrade these VOCs into less toxic, less polluting, or less dangerous compounds for health. The VOCs of interest are mainly the VOCs related to construction and decorative materials, for example paints, plywood panels, MDF (Medium-density fibreboard), carpets, . . . .

The VOC may be selected from formaldehyde, heptane, hexane, benzene, toluene, ethylbenzene, acetaldehyde, methylethylketone, limonene, pinene, xylene methanol, butanol, ethylacetate, dioctylphtalate, dichloroethane, trichlorethylene, tetrachloroethylene, trichloroethylene, and combinations thereof.

In one preferred embodiment the VOC is selected from the group consisting of formaldehyde; acetaldehyde; BTEX such as benzene, toluene, ethylbenzene, xylene; tetrachloroethylene, trichloroethylene and combinations thereof. In a preferred aspect, the VOC is formaldehyde.

As used herein, the indoor air of buildings is not specifically restricted to any type of buildings and refers to any interiors, closed spaces, such as for example houses, offices, vehicle interior, ventilation duct, performance halls (theatres, . . . ), libraries, furniture stores (shoes, textiles, . . . ), schools, hospitals, nursery . . . .

As used herein, the surface referred to is not specifically restricted and depending of the interior wherein it is desired to reduce the VOC concentrations, the surface may be for example paper, plasterboard, glass, metal, wood, textile, a polymer (polycarbonate (PC), polyamide (PA), polyester (PES), polypropylene (PP), polyethylene (PE), polyvinyl chloride (PVC), polystyrene (PS), . . . ), aluminium, steel, stainless steel, alloys, titanium.

The microorganism used in the present invention is Pseudomonas putida. Many other microorganisms, including bacteria, yeasts or fungi, are known to degrade formaldehyde. Beyond formaldehyde, Pseudomonas putida is a very versatile and widely used strain for degrading persistent organic compounds (Yonemitsu et al (2016) Biodegradation of high concentrations of formaldehyde by lyophilized cells of Methylobacterium sp. FD1, Bioscience, Biotechnology, and Biochemistry, Vol. 80, No. 11, 2264-2270). In a preferred embodiment the microorganism is Pseudomonas putida ATCC 47054. This specific strain exhibits an enzymatic pool which includes two formaldehyde dehydrogenases and two formate dehydrogenase complexes that allow to convert formaldehyde into CO₂ (Roca et al, Physiological responses of Pseudomonas putida to formaldehyde during detoxification, Microbial Biotechnology (2008) 1(2), 158-169).

The microorganism as used according to the invention can be cultivated continuously or discontinuously for example in a batch process or in a fed batch or repeated fed batch process (Filloux and Ramos (2014), Pseudomonas Methods and Protocols, In: Methods in Molecular Biology, 1149, DOI: 10.1007/978-1-4939-0473-0). The culture medium that is to be used must satisfy the requirements of the particular strains in an appropriate manner. An example of fermentation conditions of Pseudomonas putida ATCC 47054 is given in the experimental part.

One advantage of the use of Pseudomonas putida ATCC 47054 for the reduction of formaldehyde concentration in indoor air is the degradation of formaldehyde into non-toxic or less polluting compounds. Pseudomonas putida ATCC 47054 will degrade the formaldehyde into carbon dioxide and water and will not produce methanol.

One advantage of the present method is the incorporation/encapsulation of whole microorganism of Pseudomonas putida in the sol-gel matrix, compared to extracted and purified enzymes. In a preferred embodiment the whole microorganism of Pseudomonas putida is disrupted whole microbial cells of Pseudomonas putida. In one embodiment the whole microorganism of Pseudomonas Putida is a fresh crude lysate of Pseudomonas putida cells. In one preferred embodiment the whole microorganism of Pseudomonas putida that is encapsulated in a sol-gel matrix is freeze-dried (lyophilized) water dispersible particles of whole cells of Pseudomonas putida. In another preferred embodiment the whole microorganism of Pseudomonas putida that is encapsulated in a sol-gel matrix is freeze-dried (lyophilized) water dispersible particles of crude cells lysate of Pseudomonas putida. As used herein crude cells lysate or crude lysate refers to a crude extract of disrupted whole microbial cells of Pseudomonas putida wherein no purification has been performed but a preparation has been made to increase degradation of the VOC and reduce potential proliferation. The use of a lysate is preferred in order to obtain an efficient access to the degradative enzymes. The lysate may be obtained by any method known in the art, such as mechanical lysis (for example with a french press), a enzymatic lysis, a chemical lysis. In a preferred embodiment the lysis is obtained via a ultrasonic grinding. An example of experimental protocol of preparation and preservation of lyophilized whole cells or cells lysate of Pseudomonas putida ATCC 47054 is given in the experimental part. In the lyophilized state, the microorganism is biologically inactivated, but enzyme activity will be recovered after re-hydration. The lyophilized cells lysate may be kept during 4 to 5 weeks (temperature between 18 and 25° C., preferably at 20° C.) without experiencing significant loss of the enzyme activity.

In the present method, the whole microorganism of Pseudomonas putida is encapsulated into a sol-gel matrix. The sol-gel matrix must be biocompatible with the encapsulated cells of Pseudomonas putida and must ensure the enzyme activity of the encapsulated cells of Pseudomonas putida. The sol-gel matrix may be obtained by any method commonly known in the art and as disclosed in “Biocompatible Sol-Gel Route for Encapsulation of Living Bacteria in Organically Modified Silica Matrixes” (Chem. Mater., 2003, 15, 3614-3618). The sol-gel matrix is obtainable from sol-gel precursors comprising at least one hydrophobic precursor and at least one hydrophilic precursor.

The at least one hydrophobic precursor may comprise: TEOS (tetraethoxysilane), MTES (methyltrimethoxysilane), TMOS (tetramethylorthosilicate), MTMS (methyltrimethoxysilane), VTES (vinyltriethoxysilane), VTMS (vinyltrimethoxysilane), and combinations thereof. In one preferred embodiment, the at least one hydrophobic precursor comprises, consists essentially of and more preferably consists of TEOS (tetraethoxysilane).

The at least one hydrophilic precursor may comprise: GPTMS/GLYMO ((3-glycidyloxypropyl) trimethoxysilane), APTES (3-aminopropyl (triethoxysilane), MEMO (methacryloxypropyl trimethoxysilane), NAMS (N-(2-aminoethyl)-3-aminopropyltrimethoxysilane), APTMS (3-aminopropyl (trimethoxysilane), and combinations thereof. In one preferred embodiment, the at least one hydrophilic precursor comprises, consists essentially of and preferably more consists of GPTMS (3-glycidyloxypropyl) trimethoxysilane.

In addition to the at least one hydrophobic precursor and the at least one hydrophilic precursor, the sol-gel precursors may further comprise compounds selected from: crosslinking compounds to allow reticulation at room temperature, notably PVOH (poly (vinyl alcohol)) and/or PEG (polyethylene glycol), acid compounds notably acetic acid, sulfuric acid, hydrochloric acid, nitric acid and combinations thereof.

In one preferred embodiment the sol-gel precursors comprise, consist essentially of and more preferably consist of GPTMS, TEOS and PVOH.

The weight ratio of the at least one hydrophobic precursor to the at least one hydrophilic precursor is comprised in the range between 50/50 and 85/15, more preferably in the range between 60/40 and 70/30.

The sol-gel matrix may be obtained by mixing the sol-gel precursors in water at room temperature (18-30° C.). The sol-gel precursors may be mixed in a solvent other than water. The solvent may comprise: water, ethanol, isopropanol, ethanol, dioxolane, butyl acetate, butyl propionate and mixtures thereof. When the sol-gel precursors have just been mixed into solvent (notably water) thus forming a starting solution, the sol-gel precursors may make up ≥15%, ≥20%, ≥25% or ≥30% and/or ≤50%, ≤45% or ≤40% by weight of the starting solution. The dry weight of the at least one hydrophobic precursor and the at least one hydrophilic precursor in the sol-gel matrix may make up ≥5% or ≥10% and/or ≤20% or ≤15% by weight of dry matter. The pH of the sol-gel at this stage may range between 2 and 4.

In accordance with a preferred embodiment of the present invention the encapsulation of whole microorganism of Pseudomonas putida in a sol-gel matrix comprises:
providing freeze-dried water dispersible particles of Pseudomonas putida
providing a buffered sol-gel matrix at a pH of 5, and
mixing the freeze-dried water dispersible particles of Pseudomonas putida with the sol gel matrix.

After the sol-gel precursors have chemically reacted, and before the encapsulation of the water dispersible particles of Pseudomonas putida, any alcohol generated as a byproduct of the chemical reactions involved in the formation of the sol-gel matrix (for example hydrolysis and condensation of alkoxide precursors) should be removed by any suitable means, as for example by vacuum elimination by rotavapor methods. Alcohol should be removed to ensure the viability of the enzymes of the microorganism Pseudomonas putida since these enzymes are sensitive to alcohol.

A buffer is then added to the solution from which the alcohol has been removed to increase the basicity and obtain a buffered sol gel matrix at a pH of 4-5. Adjustment of the pH of the sol-gel matrix should be performed to ensure the viability of the enzymes of the microorganism Pseudomonas putida since these enzymes are sensitive to pH. The buffer may comprise: phosphate, borate, carbonate, glycine buffers, and combinations thereof. In one preferred embodiment, the buffer comprises, consists essentially of and more preferably consists of phosphate buffer.

Compounds selected from polyethylene glycol (PEG), polypropylene glycol (PPG), polyvinyl alcohol (PVOH), polycaprolactone (PCL), polytetrahydrofuran (PTHF), . . . may be added to the sol-gel matrix before the incorporation of the microorganism. Addition of these compounds may notably help to keep the hydration of the microorganism and ensure enzymatic activity and pollutant diffusion.

The encapsulation of the whole microorganism of Pseudomonas putida in the sol-gel matrix is preferably obtained by mixing the whole microorganism of Pseudomonas putida with the buffered sol-gel matrix at room temperature (15-25° C.) until homogenized. The whole microorganism of Pseudomonas putida may make up ≥20%, ≥25%, ≥30% or ≥35% and/or ≤50%, ≤45% or ≤40% by weight based on the total weight of the whole microorganism of Pseudomonas putida and the sol-gel matrix.

The sol-gel matrix with the encapsulated microorganism may be kept during at least 6 weeks at room temperature (20° C.), notably during at least 3-4 months without experiencing significant loss of the enzyme's activity.

The obtained sol-gel matrix comprising the encapsulated microorganism is then applied to a surface of interest to form a coated surface. Possible coating techniques comprise dip coating, brushing, roll coating, spraying, spin coating, flow coating, drop coating, padding, impregnation, screen-printing. The coating method will depend of the surface on which the coating is applied. For example, for paper and stainless-steel applications, spray-coating may be used, while for glass dip-coating may be preferred.

The coating may have a thickness which is ≥0.5 μm, ≥1 μm, ≥1.5 μm or ≥2 μm and/or which is ≤50 μm, ≤40 μm, ≤30 μm or ≤20 μm.

The whole microorganism of Pseudomonas putida may make up ≥20%, ≥25%, ≥ 30% or ≥35% and/or ≤50%, ≤45% or ≤40% by weight based on the total weight of the coating.

The coating may have a solvent resistance by solvent rub test using methyl ethyl ketone (MEK) which is higher than ≥100 double rubs as measured in accordance with ASTM D4752.

The coating may be replaced and/or the enzymes of Pseudomonas putida may be regenerated for example once per year in order to ensure that the coated surface continue to reduce the concentration of the VOC in air over time. The coating may be cleaned and regenerated with an aqueous solution of cofactor (notably by NADH/NAD+), notably if the surface to which the coating has been applied is subjected to external phenomena such as fouling, high temperature or detergent that might be detrimental to the enzyme activity.

After the surface has been coated with the sol-gel matrix comprising the encapsulated microorganism of Pseudomonas putida, the concentration of the VOC in air in contact with the coated surface will be reduced. In order to ensure the long term viability of the enzymes of the microorganism Pseudomonas putida, the air which is exposed to the coated surface may be at a temperature which is:
not more than 40° C., preferably not more than 37° C., more preferably not more than 30° C., and even more preferably not more than 25° C.; and/or
more than 10° C., more preferably more than 15° C., and even more preferably more than 18° C.

The concentration of the VOC, notably formaldehyde, in air in contact with the coated surface may be reduced by 10%, preferably 20%, more preferably 30%, even more preferably 40%, even more preferably 50%, even more preferably 70%, and even more preferably 80%, as compared to the concentration of said VOC in air not in contact with the coated surface.

An embodiment of the invention will now be described, by way, of example only.

### Microorganisms and Fermentation Conditions

Bacterial cells of Pseudomonas putida ATCC 47054 (ATCC, USA) were maintained in Luria Bertani agar (LBₐ) (10 g·L⁻¹ peptone, 5 g·L⁻¹ yeast extract, 10 g·L⁻¹ sodium chloride, 15 g·L⁻¹ agar) plates at 4° C. and sub-cultured monthly. Inoculum was prepared in LB broth (LB_{b}) (10 g·L⁻¹ peptone, 5 g·L⁻¹ yeast extract, 10 g·L⁻¹ NaCl) and kept overnight at 27° C., with constant agitation at 150 rpm. Cells were harvested by centrifugation (8000 rpm, 4° C., 15 min), rinsed and re-suspended in sterile water to obtain a suspension with optical density (OD600) of 1 (SYNERGY H1M, SioSPX). Fermentations were started by transferring 1 mL cells suspension (OD600=1) to 100 mL fresh LB_{b} medium (in 250 mL Erlenmeyer flask) placed at 27° C. and 150 rpm. Fermentations were stopped after about 17 h, and cells were recovered by centrifugation. Flasks containing formaldehyde solution and LB broth without the bacterial culture were used as controls to confirm any possible formaldehyde volatilization.

### Preparation and Preservation of Lyophilized Whole Cells

Microbial cells were harvested after 17 h of exponential growth phase by centrifugation at 8000 rpm at 4° C. for 15 min. Cells were suspended in carbonate buffer pH 10 (50 mM NaHCO₃, 100 mM NaOH), and the solution was put in a −45° C. isopropanol-containing bath in a HETOFRIG (HETO, Denmark) for quick freezing. After 12 h, cells were placed in the freeze dryer during 48 h. Lyophilized cells were preserved at ambient (18-20° C.) in a desiccator.

### Preparation and Preservation of Cells Lysate

Three different methods were used for the preparation of bacterial cells lysate. After lysis, cells extract was quickly transferred to a −45° C. isopropanol-containing bath HETOFRIG (HETO, Denmark). After 12 h, cells extract was place in the freeze dryer during 48 h. Lyophilized cells were preserved at ambient temperature (18-20° C.) in a desiccator.
Method 1: Cells recovered by centrifugation from the bacterial cultures after 17 h fermentation were resuspended in carbonate buffer pH 10 (50 mM NaHCO₃, 100 mM NaOH). The mixture was divided in 2 mL samples, kept in an ice bath, and disrupted using an ultra-sonicator Hielscher UP50H (VWR, Belgique) with a sonotrode MS1 (Ø=1 mm; length=80 mm). Sonication was performed within 5 cycles of 30 seconds ON and 30 seconds OFF with amplitude 80%, keeping cells flasks in the cold to avoid proteins precipitation.
Method 2: Cells lysis conditions were firstly optimized for bacteria cells using an optimized experimental design. Concentrations of phenylmethanesulphonylfluoride (PMSF) and Triton X-100 detergent, and sonication steps (number of cycles, cycle time, amplitude) were optimized in order to maximize proteins extraction and formaldehyde degradation ability. Culture samples of 20 mL were harvested by centrifugation (8000 rpm at 4° C. for 15 min) at the end of the exponential growth (after 17 h of fermentation), re-suspended in bicarbonate buffer (pH 10) (50 mM NaHCO₃, 100 mM NaOH), and kept at −80° C. Optimized cells lysis was performed as follows: After thawing, 100 μL of PMSF (0.5 mM) were added to the pellet and incubated during 2 h at 37° C. After incubation, 100 μL of Triton X-100 (0.25%) were added and the mixture was incubated at room temperature for 1 h. Finally, mixtures were ice-jacketed, and sonicated using an ultra-sonicator Hielscher UP50H (VWR, Belgique) with a sonotrode MS1 (Ø=1 mm; length=80 mm), within 7 cycles of 15 seconds ON and 15 seconds OFF with amplitude 80%.
Method 3: Bacterial cells were harvested through centrifugation (8000 rpm at 4° C. for 15 min) at the end of the exponential growth (after 17 h of fermentation) and re-suspended in bicarbonate buffer (pH 10) (50 mM NaHCO₃, 100 mM NaOH). Cells were lysed by three passages through a French Press (Thermo, Belgium) at 500 psi.

### Sol-Gel Coatings Preparation

Two different sol-gel coating preparations were prepared with the amounts of the reactants expressed in Table 1. Each sol-gel coating preparation was prepared by combining the reactants in water at room temperature (18-25° C.). Water and acid additions are carried out with stirring until homogeneity and progressive reaching of the conditions. The whole solution is then stirred for 24 hours for hydrolysis and condensation of the precursors, typical pH is about 2.5. The obtained solution can be stored in a fridge. PVOH addition in the compositions allows it to reticulate at room temperature.

TABLE 1

| Reactants | Composition | Composition |
|---|---|---|
| (g) | Ref: 90-1 | Ref: 90-3 |
| TEOS | 90 | 90 |
| GPTMS | 30 | 51.2 |
| PVOH | 24 | 24 |
| H₂O | 360 | 360 |
| CH₃COOH | 12 | 12 |

Key:
TEOS = tetraethylorthosilicate (≥99%, GPR RECTAPUR ®);
GPTMS = (3-glycidyloxypropyl)trimethoxysilane (≥97% Alfa Aesar);
PVOH = Polyvinyl alcohol (MW. 22.000 ≥98%, hydrolysed VWR) (PVOH 88% hydrolized, 22000 Mw, diluted in distilled water 20% - quantity is of the diluted PVOH);
H₂O = distilled water;
CH₃COOH = Acetic acid (100% NORMAPUR ® VWR)

### Microorganisms Incorporation

Freeze-dried (lyophilized) water dispersible particles of crude cells lysate of Pseudomonas putida ATCC 47054 prepared as described in paragraphs and were incorporated into the sol-gel coatings preparation according to the following procedure and in accordance with the quantities specified in the Table 2 below.

During the previous step of sol-gel preparation, alcohol was generated by the chemical reaction. This alcohol is removed using a ROTAVAPOR at 50° C. for 7 min 30 s, and then, a phosphate buffer is added to lower the acidity to at least pH 5, and Polyethylene glycol (PEG 600 Mw, diluted at 20 wt % in phosphate buffer (PBS)), is incorporated to increase the hydrophilicity of final coating (incorporated at 2.5% of TEOS molecular weight). The final step is the incorporation of microorganisms and homogenization of the composition, the microorganism is incorporated as previously prepared (as described above), at a ratio of 30 or 40 wt % of sol-gel dry content. Quantities of buffer, PEG (Polyethylene glycol MW 600 VWR) and microorganisms are adjusted to the dry weight of the sol-gel.

TABLE 2

| | | | | | | Sol-gel + | | |
|---|---|---|---|---|---|---|---|---|
| | Sol-gel | R1 | | PEG | Phosphate | buffer | μorganismes | |
| Series | composition | (g) | T (g) | (g) | buffer (g) | (g) | % | (mg) |
| 1 | 90-3 | 6.6 | 24.0 | 8.7 | 15.3 | 30.6 | 40 | 1285 |
| 2 | 90-3 | 10.0 | 20.8 | 0 | 20.8 | 30.8 | 40 | 1236 |
| 3 | 90-1 | 8.8 | 12.7 | 8.7 | 4.0 | 21.5 | 30 | 684 |
| 4 | 90-1 | 12.1 | 9.7 | 0 | 9.7 | 21.8 | 30 | 659 |
| 5 | 90-3 | 11.0 | 19.5 | 0 | 19.5 | 30.5 | 30 | 905 |
| 6 | 90-1 | 9.7 | 11.8 | 0 | 11.8 | 21.5 | 40 | 883 |
| 7 | 90-3 | 6.6 | 23.8 | 8.7 | 15.1 | 30.4 | 30 | 943 |
| 8 | 90-1 | 8.2 | 13.2 | 8.7 | 4.5 | 21.4 | 40 | 869 |

In the above table, R1 is the residue quantity from distillation, PEG is a PEG 600 Mw. T corresponds to the quantity of PEG and phosphate buffer quantity added. The column Sol-gel + buffer corresponds to the R1 + T quantity. When PEG is incorporated (series 1, 3, 7 and 8) it is firstly diluted into phosphate buffer and the solution is adjusted in buffer afterward

Coating Application onto Substrate

The microorganisms incorporated into the sol-gel coatings preparation as described above were coated onto paper and steel according to the following procedure and thickness measurements were measured on steel as reported in Table 3 below.

No further dilution was made for spray application on paper (5*25 cm) and steel (5*25 cm) Spray application: P=2 bars, d=20 cm and number of passes is 4, 6 or 8. Dry in the air for 24 h before the activity tests and one week for the mechanical tests.

TABLE 3

| | Average thickness of the coating (μm) | | |
|---|---|---|---|
| Series | 4 spray passes | 6 spray passes | 8 spray passes |
| 1 | 3.2 | 5.3 | 2.9 |
| 2 | 4.8 | 8.1 | 10.2 |
| 3 | 1.6 | 6.1 | 8.3 |
| 4 | 2.7 | 4.5 | 4.2 |
| 5 | 2.0 | 2.6 | 1.7 |
| 6 | 2.4 | 2.3 | 8.5 |
| 7 | 9.0 | 10.6 | 21.6 |
| 8 | 2.9 | 0.7 | 0.7 |

While no detailed results are presented herein, the microorganisms incorporated into the sol-gel coatings preparation as described in the previous paragraph were also used for dip coating with the following procedure: immersion time=30 sec and v=400 mm/min) on glass (circle diameter 1 cm).

### Formaldehyde Degradation in Solution

Formaldehyde degradation tests in solution were performed on the coated paper obtained after 6 spray passes as described above. Formaldehyde degradation tests in solution were carried out in 20 mL flasks using 4 mL of reaction mixture composed of: 1 mM formaldehyde, bicarbonate buffer (pH 10) (50 mM NaHCO₃, 100 mM NaOH) and in some cases 1 mM NAD⁺ or 1 mM NADH. Each sample of the eight series is cut to size (1×1 cm²) and 6 samples of 1×1 cm² are immersed in the reaction mixture which was kept at room temperature and constant agitation of 200 rpm. Samples were taken throughout the reaction time for formaldehyde quantification.

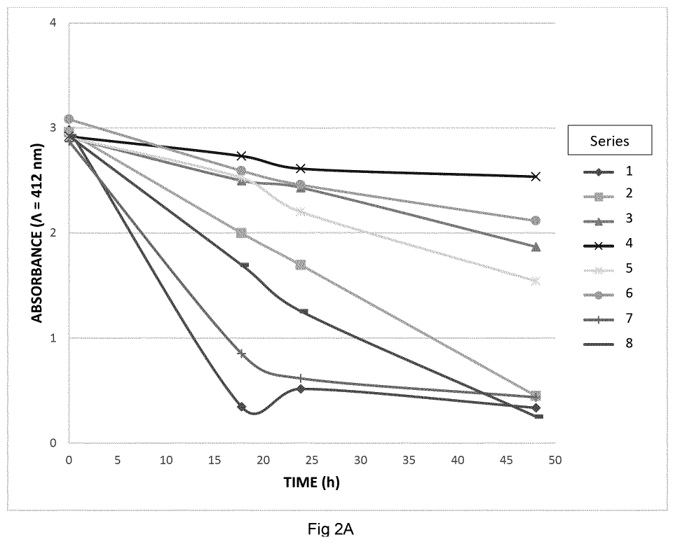

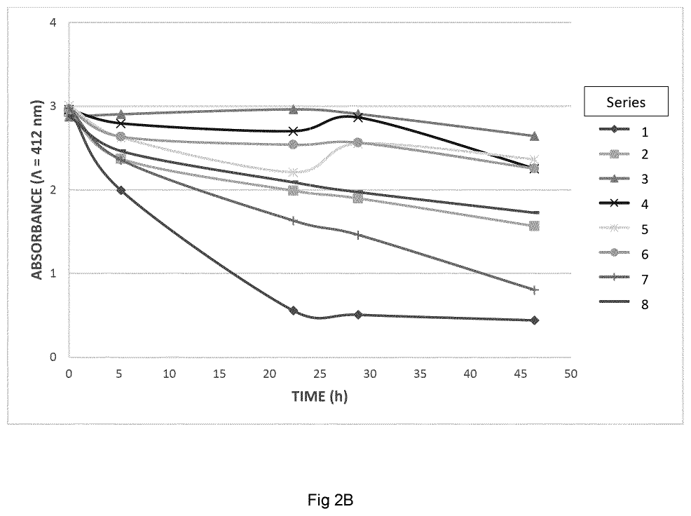

Formaldehyde was determined by visible spectrophotometry using the Nash's reagent (150 g·L⁻¹ ammonium acetate, 3 mL·L⁻¹ acetic acid, 2 mL·L⁻¹ acetylacetone). 150 μL of sample were mixed with 150 μL of the reagent. For higher formaldehyde concentrations, samples were diluted in ultra-pure water before adding NASH reagent. The mixture was incubated for 10 min at 60° C. and after cooling-down samples, absorbance was measured at 412 nm in a 1 cm cell against a reagent blank, using a Spectrophotometer SYNERGY H1M (BioSPX). The kinetics of the formaldehyde degradation is represented in FIGS. 2. In FIG. 2A the kinetics of degradation of formaldehyde by cells immobilized in sol-gel deposited on paper was performed after 24 h storage at room temperature. In FIG. 2B the kinetics of degradation of formaldehyde by cells immobilized in sol-gel deposited on paper was performed after 5 weeks storage at room temperature. As can be seen in FIG. 2A, series 2, 8, 1, and 7 allow to consume all the formaldehyde in solution, 1 and 7 having a quicker kinetic. Series 1 and 7 correspond to higher amount of GPTMS and addition of PEG. As can be seen in FIG. 2B, after 5 weeks, series 1 and 7 have a good formaldehyde degradation, with quicker kinetic for 1, corresponding to 40% of microorganisms in coating. In conclusion better results are obtained with higher amount of GPTMS in the matrix, addition of PEG and 40% of microorganisms.

### Formaldehyde Degradation Tests in the Gas Phase

Test 1: The gas phase tests were carried out using a semiconductor sensor sensitive specifically to formaldehyde. The operating principle of this sensor is based on the variation of the electrical resistance of a semiconductor material in the presence of formaldehyde. The electrical resistance variation is proportional to the formaldehyde concentration. The sensor resistance increases when the formaldehyde concentration increases.

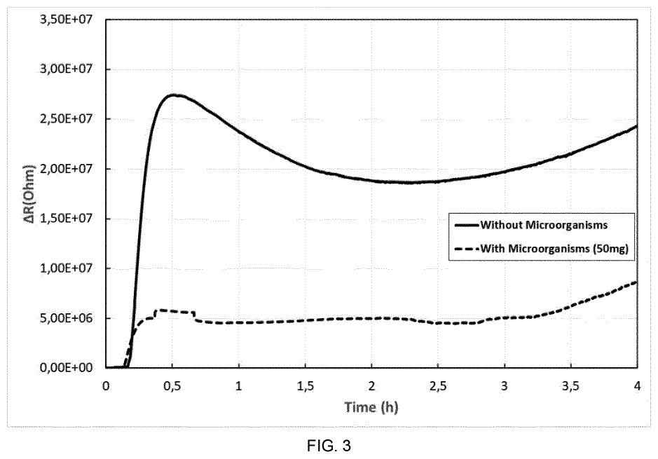

The test consisted in putting 50 mg of powdered microorganism (P Putida ATCC 47054, lyophilized cells lysate) in a 100 cm³ glass cell with a flow of humidified synthetic air HR=50%. After about ten minutes of stabilization of the sensor resistance, formaldehyde (˜700 ppb) is injected for 1 min then the cell is closed (static) and the resistance is followed as a function of time as can be seen in FIG. 3.

A reference test without microorganism was also carried out. The results shown in FIG. 3 show a degradation of formaldehyde in the presence of the microorganism, the sensor detects much less formaldehyde.

Initial testings on surfaces coated with the sol-gel matrix comprising the encapsulated microorganism of Pseudomonas putida showed a decrease of formaldehyde concentration.
Test 2: Sol-gel 90-3 was prepared (90 g TEOS with 51.2 g GPTMS has been mixed then 2 g of 20% PVOH solution was added and finally 360 g of demineralized water and 12 g of acetic acid were added under agitation). This sol-gel is stirred for 24 hours. After determination of the dry extract, elimination of the alcohol, addition of the phosphate buffer, the PEG and the micro-organisms of Pseudomonas putida, the solution was stirred for at least 24 hours to allow a homogeneous application. TLC (Thin-Layer Chromatography) papers were then sprayed at 20 cm, at a flow rate of 15 mL/min, in 6 passes, and then dried at 20° C. for 48 h. The gas phase tests were carried out using a method according to a method close to that of the ISO 16000-23 standard.

4 sheets of functionalized TLC samples were installed in the bottom of the test chamber (volume 1.08 m³). Formaldehyde was introduced in the chamber thanks to a standard cylinder of formaldehyde at 5 ppmv concentration. Once the concentration in the chamber reached about 500 ppbv, the cylinder is disconnected from the chamber. The air in the chamber is homogenised with and fan. The formaldehyde concentration in the chamber was followed online by IMR-MS (ion molecule reaction-mass spectrometry). The IMR-MS was calibrated with a standard at 500 ppbv made from the formaldehyde cylinder.

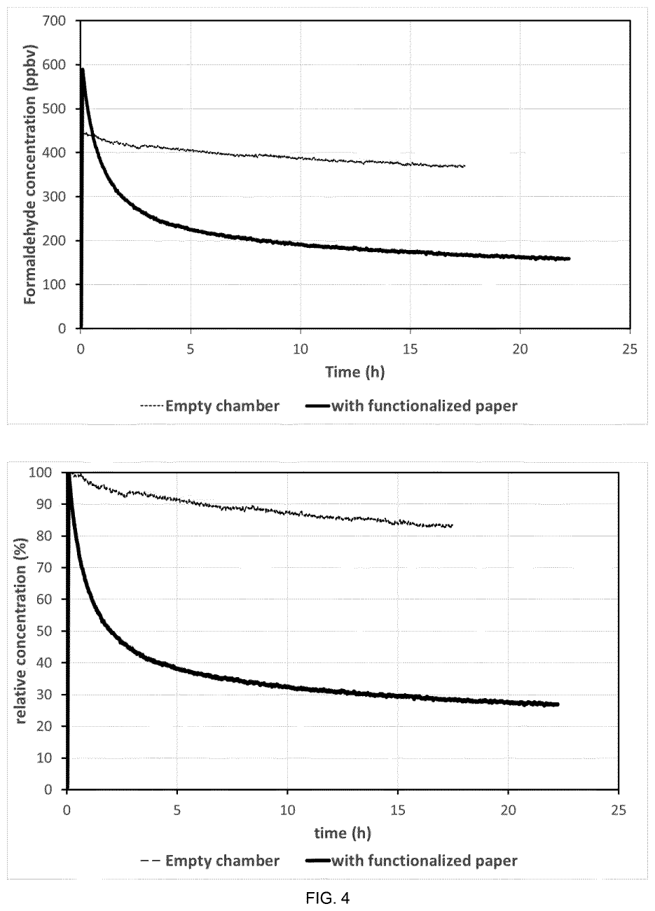

The results shown in FIG. 4 show a good degradation of formaldehyde in the presence of the sheets functionalized than in their absence.

1. A method to reduce concentration of a VOC (volatile organic compound) in air in contact with a coated surface comprising:
providing a surface
encapsulation of whole microorganism of Pseudomonas putida in a sol-gel matrix
application of the obtained sol-gel matrix to the surface to form the coated surface, and
exposing the air to the coated surface.

2. The method according to claim 1, wherein the whole microorganism is disrupted whole microbial cells of Pseudomonas putida.

3. The method in accordance with claim 1, wherein the microorganism consists of Pseudomonas putida ATCC 47054.

4. The method in accordance with claim 1, wherein the VOC is converted in to non-toxic or less polluting compound(s).

5. The method in accordance with claim 1, wherein the surface is selected from the group consisting of paper, plasterboard, glass, metal, wood, textile, aluminium, steel for example stainless-steel and alloys.

6. The method in accordance with claim 1, wherein the air which is exposed to the coated surface is air in the interior of a building, such as a house, office, vehicle interior and ventilation duct.

7. The method in accordance with claim 1, wherein the air which is exposed to the coated surface is at a temperature which is
not more than 40° C., preferably not more than 37° C., more preferably not more than 30° C., and even more preferably not more than 25° C.; and/or
more than 10° C., more preferably more than 15° C., and even more preferably more than 18° C.

8. The method in accordance with claim 1, wherein the VOC is selected from the group consisting of formaldehyde; acetaldehyde; BTEX such as benzene, toluene, ethylbenzene, xylene; tetrachloroethylene, trichloroethylene, and combinations thereof.

9. The method in accordance with claim 1, wherein the encapsulation of whole microorganism of Pseudomonas putida in a sol-gel matrix comprises:
providing freeze-dried water dispersible particles of Pseudomonas putida
providing a buffered sol-gel matrix at a pH of 5, and
mixing the freeze-dried water dispersible particles of Pseudomonas putida with the sol gel matrix.

10. The method in accordance with claim 1, wherein the sol-gel matrix is obtained from sol-gel precursors comprising at least one hydrophobic precursor and at least one hydrophilic precursor.

11. The method in accordance with claim 10, wherein the weight ratio of the at least one hydrophobic precursor to the at least one hydrophilic precursor is comprised in the range between 50/50 and 85/15, more preferably between 60/40 and 70/30.

12. The method in accordance with claim 10, wherein the sol-gel precursors comprise GPTMS, TEOS and PVOH.

13. The method in accordance with claim 1, wherein the whole microorganism of Pseudomonas putida is comprised in a quantity between 20 to 40% by weight based on the total weight of whole microorganism of Pseudomonas putida and the sol-gel matrix.

14. The method in accordance with claim 1, wherein the VOC is formaldehyde and wherein the formaldehyde is transformed in to water and carbon dioxide by exposing the formaldehyde in the air to the coated surface.

15. A method to reduce the concentration of a VOC, notably formaldehyde, in air wherein the method comprises exposing the air with encapsulated whole microorganism of Pseudomonas putida in a sol-gel matrix.
